(19) United States
(12) Patent Application Publication
(10) Pub. No.: US 2025/0050169 A1
(43) Pub. Date: Feb. 13, 2025

(54) IMAGE PROCESSING DEVICE INTERWORKING WITH WEIGHT TRAINING EQUIPMENT

(71) Applicant: LG ELECTRONICS INC., Seoul (KR)

(72) Inventors: Sungwon BEOM, Seoul (KR); Junyoung HUH, Seoul (KR); Hyunjae NOH, Seoul (KR); Changsun PARK, Seoul (KR)

(21) Appl. No.: 18/719,556

(22) PCT Filed: Apr. 28, 2023
(86) PCT No.: PCT/KR2023/005865

§ 371 Date: Jun. 13, 2024

(30) Foreign Application Priority Data
Aug. 22, 2022 (KR) 10-2022-0104886
Dec. 28, 2022 (KR) 10-2022-0187522

(51) Int. Cl. A63B 24/00; A63B 21/00; A63B 21/005; A63B 71/06

(52) U.S. Cl. CPC A63B 24/0062; A63B 21/0058; A63B 21/153; A63B 21/4033; A63B 71/0622; A63B 2024/0068; A63B 2071/0647; A63B 2220/05; A63B 2220/20; A63B 2220/807

(57) ABSTRACT

Proposed is an image processing device interworking with weight training equipment. In the image processing device, when an expert's exercise image is matched with a user's exercise image and reproduced, postures in the expert's exercise image and the user's exercise image may be synchronized and reproduced. Accordingly, it is possible to more accurately compare the exercise postures using the expert's exercise image and the user's exercise image.

### TECHNICAL FIELD

The present disclosure relates to an image processing device interworking with weight training equipment and, more particularly, to an image processing device that displays a fitness expert's exercise image and a user's exercise image in conjunction with weight training equipment.

### BACKGROUND ART

In general, weight training equipment used for strength exercise is designed so that a user repeats relaxation and contraction of muscles while pushing or pulling a lever connected to a weight having a certain unit of weight.

The weight training equipment includes an arm curl machine for biceps exercises, a chest press or butterfly machine for chest muscle, such as the pectoralis major, exercises, and a pull-up machine for shoulder and back muscle exercises.

Recently, as part of self care, home training (workout) is becoming an issue. Home training refers to exercising for health management at home, one's own “querencia (shelter or sanctuary in Spanish)”, instead of going out for a jog or visiting a fitness center. In line with this trend, not only various exercise equipment for home training have been proposed, but also a new South Korean term “Homt-jok” for people who enjoy home training has been created.

An example of weight training equipment for home training is a workout machine utilizing cable wires. Such a cable machine has a drive motor installed therein and transfers an exercise load provided from the drive motor to a user through a cable wire, allowing the user to perform strength exercises while pulling or releasing the cable wire.

Meanwhile, in weight training, a proper posture during workout can increase the efficiency of the exercise, and continuous workout with a bad posture may adversely affect the body.

In a fitness center, professional trainers correct a member's posture to guide the member to exercise in the proper posture. However, in the case of home training, since it is not possible to receive direct coaching from a professional trainer on site, various methods have been proposed to replace direct coaching.

One example is providing a fitness expert's exercise video for a specific strength exercise. That is, the process of performing a specific strength exercise by an expert is filmed as a video and provided to users who do workouts at home.

Then, a user may perform a strength exercise while photographing his/her own exercise video and comparing the expert's exercise image with the his/her own exercise image in real time as shown in FIG. 1.

Yet, exercises of experts and general users may differ in speed. For example, assuming that an expert's one exercise cycle takes 3 seconds whereas a general user's one exercise cycle takes 2 seconds, when the user checks the expert's video while exercising, the user may find that his/her posture is different from that of the expert.

In weight training, each user performing weight training may have different exercise cycles due to differences in physical conditions and muscle strength. Thus, it is undesirable for a user to exercise according to the expert's exercise cycle.

In other words, since the correct posture in one exercise process rather than one exercise cycle is recognized as an important factor in weight training, a process of synchronizing an expert's exercise image with a user's exercise image is necessary.

What can be generally considered in synchronization of images is a method of increasing or decreasing the speed of an expert's video. Assuming that an expert's one exercise cycle is 3 seconds as in the example described above, when the playback speed is increased so that one exercise cycle takes 2 seconds, an expert's exercise image and a user's exercise image coincide in time.

FIGS. 2A to 2C are views illustrating examples of movement positions over time in an expert exercise image (such as moving picture, video, etc.) and a user exercise image (such as moving picture, video, etc.). FIG. 2A is a view illustrating a user's movement distance in the user exercise image, and FIG. 2B is a view illustrating an expert's movement distance in the expert exercise image.

For example, as shown in FIG. 1, when performing a squat in a state in which an exercise load is provided, the movement distance of the user or expert is repeated at regular intervals. FIGS. 2A to 2C show the movement positions of the user and the expert when one squat exercise cycle is performed.

At this time, the movement distance is the distance that the user or expert moved in the course of weight training. For example, in the case of exercise using a cable wire, the movement distance means the distance that the cable wire is pulled out and then retracted. In other words, the movement distance is the total distance in the process of pulling out and retracting the cable wire, and in the examples illustrated in FIGS. 2A to 2C, the pulled-out and retracted positions of the cable wire are shown.

In addition, one exercise cycle refers to one repeated motion in strength exercises with cyclical characteristics, such as squat. For example, in the case of a squat, one motion of standing up and squatting down is defined as one exercise cycle.

As shown in FIGS. 2A and 2B, exercise speeds of the user and the expert, that is, periods of one cycle may be different as t_{FU} and t_{FT}, respectively, and the time to reach the peak position may also be different for the user and the expert as t_{PU} and t_{PT}, respectively.

In this regard, when the playback speed of the expert exercise video is increased to adjust the expert's exercise speed according to the user's exercise speed, a movement position of the expert is shifted as shown in FIG. 2C.

In this case, although the playback time of one exercise cycle is the same, the postures in the user's exercise image and the expert's exercise image may be different. As shown in FIGS. 2A to 2C, when speeding up the playback time, the time at the peak position in the expert's exercise image is changed to t′_{PT}, resulting in a larger deviation in the peak positions of the user's exercise video and the expert's exercise video than when the image is reproduced at normal speed as shown in FIG. 2B.

Furthermore, usually, when shooting an expert exercise video, an expert's voice guiding exercise technique is recorded together, but if the playback speed is adjusted, an unnatural voice is output, which is problematic. In addition, as previously mentioned, there is a problem in that a guide voice to be provided with a certain posture is actually output with another posture due to the adjustment of the playback speed.

### DISCLOSURE OF INVENTION

### Technical Problem

Accordingly, the present disclosure has been made keeping in mind the above problems occurring in the related art, and the present disclosure is intended to provide an image processing device interworking with weight training equipment, the image processing device being capable of synchronizing postures in an expert's exercise image and a user's exercise image before reproducing the images when playing back the expert's exercise image after matching the expert's exercise image to the user's exercise image.

An objective of the present disclosure is to provide an image processing device that allows a user to more accurately compare his/her posture with an expert's posture at a specific point in time when the user exercises while comparing an expert's exercise image with his/her own exercise image.

An objective of the present disclosure is to provide an image processing device capable of outputting an exercise guide voice provided by an expert without being damaged or deformed when playing back an expert's exercise image synchronized with a user's exercise image.

An objective of the present disclosure is to provide an image processing device that enables an exercise guide voice provided by an expert to be accurately output at the point of the corresponding posture when playing back an expert's exercise image synchronized with a user's exercise image.

An objective of the present disclosure is to provide an image processing device capable of automatically providing an expert's exercise image showing movement in the direction corresponding to the direction of a user's exercising hand when a user performs a one-hand exercise.

### Solution to Problem

In order to achieve the above objectives, according to an embodiment of the present disclosure, there is provided an image processing device interworking with weight training equipment. In the image processing device, an expert movement distance in an expert exercise image may be matched with a user movement distance and displayed, so that the expert exercise image may be displayed in synchronization with a user exercise image.

In the embodiment, an image controller may control an expert image frame to be displayed on an image display part among expert image frames constituting the expert exercise image on the basis of the user movement distance received from the weight training equipment through a communication part.

The image processing device according to the embodiment may include a data storage part in which an expert exercise image is stored.

In addition, the image processing device may include a camera module configured to capture images of a user exercising on the weight training equipment. At this time, the image controller may display a user exercise image captured by the camera module together with an expert exercise image on the image display part.

In the embodiment, the weight training equipment may transmit a user movement distance based on the user's exercise to the image processing device through a communication network.

In the embodiment, the image controller may match an expert exercise image and a user exercise image in units of one exercise cycle and display the matched image on the image display part.

In the embodiment, each expert image frame constituting the expert exercise image may include an expert movement distance of the corresponding expert image frame within one exercise cycle of an expert.

At this time, the expert movement distance may include an expert ratio. The expert ratio may be defined as a ratio of the expert movement distance of the corresponding expert image frame to the total movement distance within one exercise cycle of the expert.

The image processing device according to the embodiment may include a user movement analysis part. The user movement analysis part may extract one exercise cycle of the user on the basis of the user movement distance.

In the embodiment, the image controller may match the expert exercise image and the user exercise image in units of one exercise cycle on the basis of one exercise cycle of the user and one exercise cycle of the expert.

In the embodiment, the user movement analysis part may calculate a total movement distance within one exercise cycle of the user. The user movement analysis part may calculate a user ratio defined as the ratio of a current movement distance of the user to the total movement distance of the user.

The image controller may match an expert image frame having an expert ratio matching the user ratio to the current movement distance of the user and display the matched image on the image display part.

In the embodiment, the user movement analysis part may calculate a total movement distance within one exercise cycle of the user for each exercise cycle. The user movement analysis part may calculate a user ratio in a current exercise cycle on the basis of a total movement distance calculated in a previous exercise cycle.

In the embodiment, the user movement analysis part may extract an inflection point in a movement direction of the user from the user movement distance, and extract one exercise cycle of the user on the basis of the inflection point.

At this time, the inflection point may be formed alternately with a first inflection point in which the movement direction changes in one direction and a second inflection point in which the movement direction changes in an opposite direction to the first inflection point. The user movement analysis part may extract an interval between a pair of adjacent first inflection points as one exercise cycle of the user.

In the embodiment, the user movement analysis part may calculate a total movement distance within one exercise cycle of the user by summing a movement distance from one of the pair of first inflection points to a second inflection point, and a movement distance from the second inflection point to a remaining one of the pair of first inflection points, which determine the one exercise cycle.

The data storage part according to the embodiment of the present disclosure may store an exercise guide voice matched to a corresponding image frame to correspond to the expert movement distance.

The image controller may synchronize and output the exercise guide voice when displaying the expert exercise image on the image display part, but may output the voice in synchronization with a display of the matched image frame on the image display part.

The weight training equipment according to the embodiment of the present disclosure may provide an exercise load through a pair of cable wires. The user movement distance may include a pulled-out position of each of the pair of cable wires.

In the embodiment, the expert exercise image may include a left arm exercise image and a right arm exercise image for a same exercise type.

The image controller may display one of the left arm exercise image and the right arm exercise image on the image display part on the basis of pulled-out cable wire information received from the weight training equipment through the communication part.

### Advantageous Effects of Invention

An image processing device interworking with weight training equipment according to the present disclosure has one or more of the following effects.

First, an expert's exercise posture in an expert's exercise image and a user's exercise posture in a user's exercise image can be synchronized in a mutually matched state when synchronizing the expert's exercise image with the user's exercise image.

Second, when a user exercises while comparing an expert's exercise image with his/her own exercise image, the images are mutually synchronized on the basis of expert's and user's movement distances, so that the expert's and user's postures can be more accurately compared at a specific point in time.

Third, since an output of an exercise guide voice is not affected by changing the playback time, the guide voice can be output without damage or deformation.

Fourth, since an exercise guide image for a specific posture is synchronized and output with an expert image frame corresponding to the posture, it is possible to provide a more accurate exercise guide at the desired time point.

Fifth, in the course of a user performing a one-hand exercise, the direction of a user's hand can be automatically recognized, and an expert's exercise image showing movement in the direction corresponding to the direction of a user's exercising hand can be displayed.

### BRIEF DESCRIPTION OF DRAWINGS

FIG. 1 is a view illustrating an example in which an expert exercise image and a user exercise image are displayed;

FIGS. 2A to 2C are views illustrating a conventional method of synchronizing an expert exercise image with a user exercise image;

FIG. 3 is a view illustrating an example of operation of an image processing device and weight training equipment according to an embodiment of the present disclosure;

FIG. 4 is a control block diagram of the weight training equipment according to the embodiment of the present disclosure;

FIG. 5 is a control block diagram of the image processing device according to the embodiment of the present disclosure; and

FIGS. 6A to 9 are views illustrating a principle of synchronizing an expert exercise image with a user exercise image in the image processing device according to the embodiment of the present disclosure.

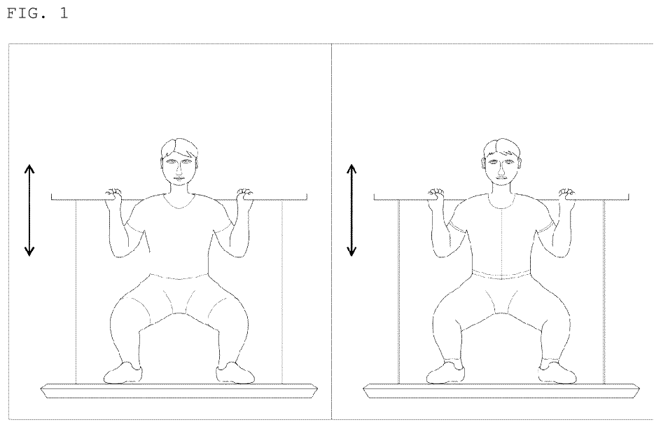

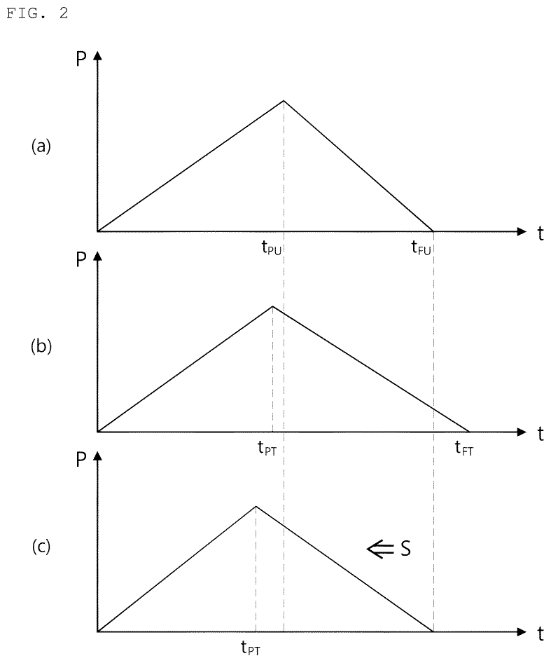

### BEST MODE FOR CARRYING OUT THE INVENTION

The present disclosure relates to an image processing device interworking with weight training equipment.

In an image processing device according to an embodiment of the present disclosure, when an expert's exercise image is matched with a user's exercise image and reproduced, postures in the expert's exercise image and the user's exercise image may be synchronized and reproduced.

### MODE FOR THE INVENTION

Advantages and features of the present disclosure, and methods of achieving them, will become clear with reference to the detailed description of the following embodiments in conjunction with the accompanying drawings. However, the present disclosure is not limited to the embodiments disclosed below and may be implemented in various different forms. The embodiments are provided to complete the disclosure of the invention and to fully inform those skilled in the art of the scope of the invention, and the present disclosure is only defined by the scope of the claims. Like reference numbers designate like elements throughout the specification.

Prior to the description of the present disclosure, terms used to describe the present disclosure are defined.

The “movement distance” in “user movement distance” and “expert movement distance” is the distance that a user or an expert moved in the process of weight training. For example, in the case of strength exercise using a cable wire 120 to be described later, the movement distance means the total distance that the cable wire is pulled out and retracted. That is, the movement distance is defined as the total distance of the distance from which the cable wire is pulled out and the distance from which the cable wire is pulled in.

In addition, one exercise cycle refers to one repeated motion in strength exercises with cyclical characteristics, such as a squat. For example, in the case of a squat, one motion of standing up and squatting down is defined as one exercise cycle.

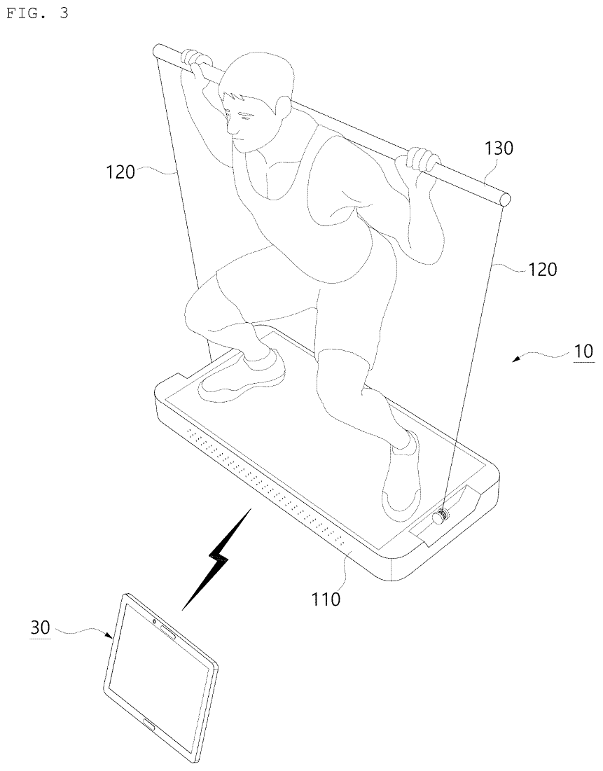

FIG. 3 is a view showing an example of operations of an image processing device 30 and weight training equipment 10 according to an embodiment of the present disclosure.

The weight training equipment 10 according to the embodiment of the present disclosure has, for example, a structure for strength exercise by pulling or releasing a pair of cable wires 120.

Referring to FIG. 3, the weight training equipment 10 may include a main body 110. In the embodiment of the present disclosure, it is taken as an example that the main body 110 has a mat shape. With the main body 110 placed on the floor indoors or the ground outdoors, a user may climb on top of the main body 110 and perform a muscle strength exercise using the pair of cable wires 120 extending from the main body 110 to the outside.

At this time, a gripping attachment for a user to grip and exercise may be connected to the pair of cable wires 120. As shown in FIG. 3, a handle bar 130 to which the cable wires 120 are connected may be installed at both ends of the main body 110. In addition, a grip such as a D-grip may be connected to each of the cable wires 120 on each side of the main body 110, and various types of gripping attachments for exercise may be connected.

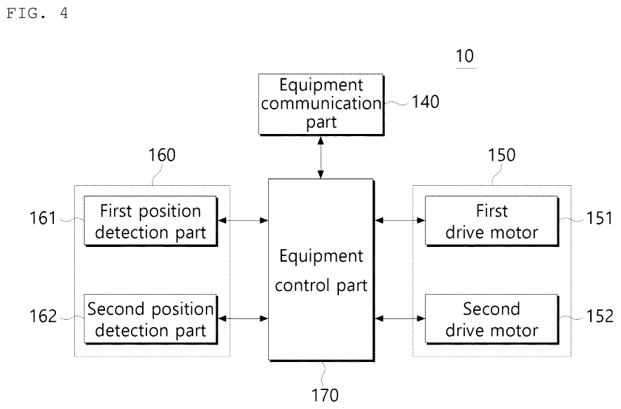

FIG. 4 is a control block diagram of the weight training equipment 10 according to the embodiment of the present disclosure.

Referring to FIG. 4, the weight training equipment 10 according to the embodiment of the present disclosure may include a load supply module 150.

The load supply module 150 according to the embodiment of the present disclosure may generate an exercise load and provide the generated exercise load to a user through the pair of cable wires 120.

In the embodiment of the present disclosure, the load supply module 150 includes a first drive motor 151 and a second drive motor 152. The first drive motor 151 and the second drive motor 152 may independently generate an exercise load and provide the generated exercise load through each cable wire 120.

Accordingly, in a state in which the first drive motor 151 and the second drive motor 152 output mutually corresponding exercise loads, a movement in which the pair of cable wires 120 are pulled and released in the same direction is possible as in the squat exercise shown in FIG. 3.

In addition, with a D-grip or the like connected to each cable wire 120, a movement in which the pair of cable wires 120 are pulled and released in opposite directions is possible. One-handed exercise using only one cable wire 120 is also possible.

Although FIG. 3 shows an example in which the first drive motor 151 and the second drive motor 152 independently generate exercise loads, it is also possible to use a single drive motor and a differential mechanism to provide equal exercise load through the pair of cable wires 120.

Meanwhile, the weight training equipment 10 according to the embodiment of the present disclosure may include a position detection part 160.

The position detection part 160 may detect the pulled-out positions of the cable wires 120. As previously mentioned, since the first drive motor 151 and the second drive motor 152 are independently connected to the respective cable wires 120, the position detection part 160 may include a first position detection part 161 and a second position detection part 162.

At this time, the first position detection part 161 detects the pulled-out position of any one cable wire 120 of the pair of cable wires 120 while the second position detection part 162 detects the pulled-out position of the other cable wire 120 of the pair of cable wires 120.

As one example, the first position detection part 161 and the second position detection part 162 may detect the pulled-out positions of the cable wires 120 by directly or indirectly detecting rotation amounts of the first drive motor 151 and the second drive motor 152. For example, the first position detection part 161 and the second position detection part 162 may detect the rotations of the first drive motor 151 and the second drive motor 152, respectively.

As another example, the load supply module 150 according to the embodiment of the present disclosure may include a first drive system and a second drive system for transmitting rotational forces of the first drive motor 151 and the second drive motor 152 to the cable wires 120, respectively. For example, each of the first drive system and the second drive system may include a bobbin, a timing belt, and a transmission pulley.

The transmission pulley may be connected to the first drive motor 151 and the second drive motor 152 through the timing belt, and rotate in synchronization with the rotations of the first drive motor 151 or the second drive motor 152. The bobbin is coaxially coupled with the transmission pulley in a state in which the cable wire 120 is wound, and rotates in synchronization with the rotation of the transmission pulley so as to transmit the exercise load of the first drive motor 151 or the second drive motor 152 transmitted through the timing belt and transmission pulley to each cable wire 120.

At this time, the first position detection part 161 and the second position detection part 162 may detect the pulled-out position of each cable wire 120 by detecting the movement of the timing belt in the forward and reverse directions according to the rotations of the first drive motor 151 and the second drive motor 152.

Configurations of the first position detection part 161 and the second position detection part 162 according to the embodiment of the present disclosure are not limited to those of the above-described embodiment, and may be configured in various forms to detect the positions of the cable wires 120 moving according to the user's motion.

The weight training equipment 10 according to the embodiment of the present disclosure may include an equipment communication part 140. As an example, the equipment communication part 140 may be connected to the image processing device 30 through a communication network such as Bluetooth or Wi-Fi.

The weight training equipment 10 according to the embodiment of the present disclosure may include an equipment controller 170.

The equipment controller 170 may control all functions of the weight training equipment 10 according to the embodiment of the present disclosure. For example, the equipment controller 170 may control the first drive motor 151 and the second drive motor 152 so that the exercise load is output to a target load set by the user.

In the embodiment of the present disclosure, the equipment controller 170 transmits the user's movement distance based on the pulled-out position of each cable wire 120 detected by the first position detection part 161 and the second position detection part 162 to the image processing device 30 through the equipment communication part 140, which will be described later.

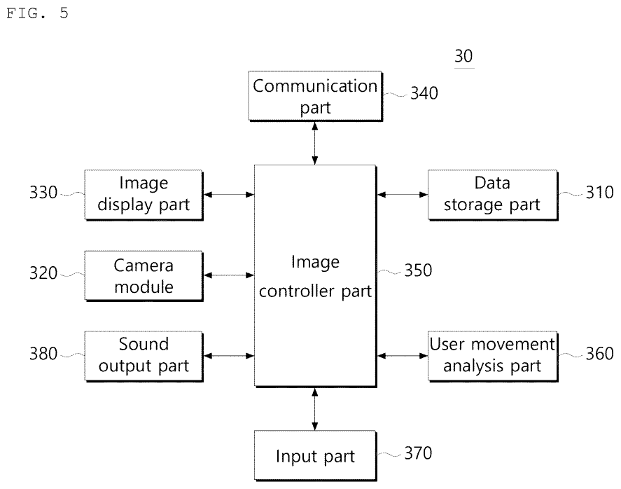

FIG. 5 is a control block diagram of the image processing device according to the embodiment of the present disclosure.

Referring to FIG. 5, the image processing device 30 according to the embodiment of the present disclosure may include a data storage part 310.

In the data storage part 310, expert exercise videos may be stored. For example, expert exercise videos may be stored for each type of exercise. That is, expert exercise videos in which images of experts exercising for various exercise types, such as a squat, deadlift, bench press, two-handed or one-handed exercise using a barbell, are recorded may be stored in the data storage part 310.

In addition, expert exercise images captured in various directions, such as images taken from the front, from the side, and from a forward 450 direction, may be stored for each exercise type.

The image processing device 30 according to the embodiment of the present disclosure may include a camera module 320. For example, the camera module 320 may capture a user exercising in the weight training equipment 10.

In the embodiment of the present disclosure, the camera module 320 includes a CMOS image sensor and is installed in front of the image processing device 30 to capture a motion image of a user. As another example, a separate camera module 320 for capturing images may be connected to the image processing device 30 so that the image processing device 30 may receive images captured from that separate camera module 320 through wired or wireless communication.

The image processing device 30 according to the embodiment of the present disclosure may include an image display part 330. For example, when the image processing device 30 according to the embodiment of the present disclosure is provided in the form of a tablet, an image display part 330 may be provided on the front surface of one side of the image processing device 30.

The image display part 330 may be made of an LCD type or an OLED type, and may display an expert exercise image and a user exercise image captured by the camera module 320 on the screen under the control of an image controller 350 to be described later.

The image processing device 30 according to the embodiment of the present disclosure may include a communication part 340.

For example, the communication part 340 may be connected to the weight training equipment 10 through a communication network such as Bluetooth or Wi-fi. In addition, the communication part 340 may receive the user's movement distance from the weight training equipment 10.

The image processing device 30 according to the embodiment of the present disclosure may include the image controller 350. The image controller 350 may include a hardware component and a software component for controlling the functions of the image processing device 30.

For example, the image controller 350 may include an application program provided by the weight training equipment 10 according to the embodiment of the present disclosure. At this time, the application program may be installed in the image processing device 30 such as a tablet and operate in conjunction with an operating system of the tablet.

In the embodiment of the present disclosure, the image controller 350 displays expert exercise images stored in the data storage part 310 and user exercise images captured by the camera module 320 on the image display part 330, so that the user may exercise while comparing his/her current exercise image with an expert exercise image.

For example, when the user selects, through an input part 370, the type of exercise he/she wants to do and the direction of the movement of an expert he/she wants to see, the image controller 350 may retrieve an expert exercise image corresponding to the user-selected exercise type and shooting direction from the data storage part 310, and display the retrieved image on the image display part 330 together with a user exercise image.

For example, the image controller 350 controls the expert's movement distance in the expert's exercise image to be matched with the user's movement distance in the user's exercise image and displayed on the basis of the user movement distance received through the communication part 340.

At this time, for example, the image controller 350 controls an expert image frame to be displayed on the image display part 330 among expert image frames constituting an expert exercise image on the basis of the user movement distance received through the communication part 340.

For example, on the basis of the user movement distance, the image controller 350 may display the expert exercise image and the user exercise image on the image display part 330 in units of one exercise cycle.

To be specific, an image of a user exercising using the weight training equipment 10 is captured by the camera module 320 and displayed on the image display part 330 in real time. In addition, the user's movement distance based on the pulled-out position of each wire 120 detected by the first position detection part 161 and the second position detection part 162 of the weight training equipment 10 is also transmitted to the image processing device 30 through the equipment communication part 140.

Accordingly, the image controller 350 selects an expert image frame corresponding to the user's exercise distance received in real time through the communication part 340, and displays the selected expert image frame on the image display part 330 to match the expert's exercise posture to the user's exercise posture.

In other words, by selecting and displaying an expert video frame within the expert exercise video according to the user's current movement distance transmitted in real time without adjusting the playback time of the expert exercise video, the exercise postures of the user and the expert are matched.

For example, each expert image frame constituting the expert exercise image according to the embodiment of the present disclosure may include information about an expert movement distance of a corresponding expert image frame within one exercise cycle of the expert.

Hereinafter, an example of a method of including information about expert movement distance in each expert image frame of expert exercise video in the embodiment of the present disclosure will be described.

When the expert exercise video recording the expert's exercise process is extracted, images corresponding to one exercise cycle are extracted while viewing the expert exercise video.

When the images corresponding to one exercise cycle are extracted, all frames in the images, that is, all expert image frames are extracted.

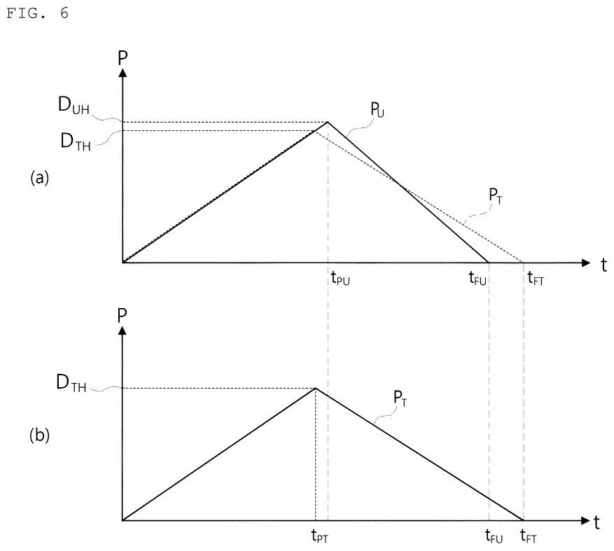

FIGS. 6A and 6B are examples of graphs showing the positions of the cable wires 120 over time during one exercise cycle of a user in a user exercise image and one exercise cycle of an expert in an expert exercise image. FIG. 6A is a graph showing the position (Pᵤ) of the user's cable wire 120, and FIG. 6B is a graph showing the position (P_{T}) of the expert's cable wire 120.

Referring to FIG. 6B, Time T_{FT} was required for one exercise cycle of the expert, and the distance D_{TH} was reciprocated based on the position of the wire, so the total movement distance was 2D_{TH}.

In addition, assuming that the expert exercise video is recorded at 30 fps, the total number of expert image frames constituting one exercise cycle is 30 fpsXt_{FT}.

Assuming that the expert performed a uniform motion, the expert's movement distance in one expert image frame is 2D_{TH}/(30 fpsXt_{FT}). That is, the expert moves 2D_{TH}/(30 fpsXt_{FT}) for every expert image frame.

Thus, assuming that 2D_{TH}/(30 fpsXt_{FT}) is FD, the movement distance in the first expert image frame is FD, the movement distance in the second expert image frame is 2FD, and the movement distance in the nth expert image frame is nFD, and these values become information about the expert movement distance.

At this time, as shown in FIGS. 6A and 6B, due to differences in physical conditions such as height between the expert and the user, the expert's exercise movement and the user's exercise movement have different total movement distances within one exercise cycle.

For example, the total expert movement distance 2D_{TH} and the total user movement distance 2D_{UH} may be different from each other as the expert moves back and forth over the movement distance D_{TH} while the user moves back and forth over the movement distance D_{UH}.

Thus, in the case of recording expert movement distance information in the expert image frame as the expert movement distance itself, synchronization may not be achieved due to the difference between the total expert distance 2D_{TH} and the total user movement distance 2D_{UH}.

Accordingly, in the embodiment of the present disclosure, the expert movement distance is determined by an expert ratio, which is defined as a ratio of the expert's movement distance in the corresponding expert image frame to the total movement distance within one exercise cycle of the expert.

Thus, the expert movement distance in the first expert video frame is FD/2D_{TH}, the expert movement distance in the second expert video frame is 2FD/2D_{TH}, and the expert movement distance in the nth expert video frame is nFD/2D_{TH}, and this expert ratio may be stored in each expert image frame as information on the expert movement distance.

Hereinafter, a method of synchronizing expert exercise images with user exercise images will be described on the basis of the expert exercise video as above and the user movement distance received through the communication part 340.

The image processing device 30 according to the embodiment of the present disclosure may include a user movement analysis part 360. For example, the user movement analysis part 360 may extract one exercise cycle of the user based on the user's movement distance.

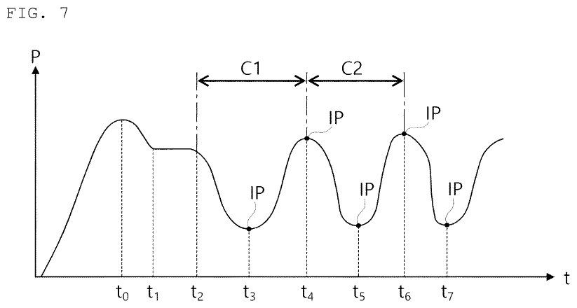

FIG. 7 is a graph showing the pulled-out position of the cable wire 120 detected by the first position detection part 161 or the second position detection part 162 when the user exercises in the weight training equipment 10 according to the embodiment of the present disclosure. That is, the graph of FIG. 7 shows an example of performing squats with the handle bar 130 connected to the cable wire 120 positioned at the back of the neck.

Referring to FIG. 7, when the user pulls the handle bar 130 for a squat, the pulled-out position of the cable wire 120 increases. In addition, at time to, when the handle bar 130 moves over the user's head and toward the back of the neck, the pulled-out position increases until time t₁ and then stops at the back of the neck.

In the embodiment of the present disclosure, when the cable wire 120 does not move at a certain position and is maintained for a predetermined time, it is recognized that the preparation posture for preparing for exercise is completed, and the exercise load is applied according to the target load set by the user to start the exercise.

For example, as shown in FIG. 7, exercise preparation is completed between time ti and time t₂, and exercise may start from time t₂ in a state where the target load is applied.

Like the expert's squat, the user's squat also shows a form of repeating a certain cycle. As shown in FIG. 7, as the user repeats the sitting posture and standing posture based on the pulled-out positions of the cable wires 120, the pulled-out positions of the cable wires 120 show a periodic change with an inflection point (IP) at a high point and a low point.

Thus, in the embodiment of the present disclosure, the user movement analysis part 360 extracts one exercise cycle C1 and C2 of the user on the basis of inflection points (IP) based on the pulled-out positions of cable wires 120. In FIG. 7, the inflection points (IP) are detected at times t₃, t₄, t₅, t₆, and t₇ as an example.

As previously described, the image controller 350 matches the expert exercise image with the user exercise image in units of one exercise cycle on the basis of the user's one exercise cycle and the expert's one exercise cycle.

To be specific, the user movement analysis part 360 may calculate the total movement distance within one exercise cycle of the user. The user movement analysis part 360 may also calculate a user ratio defined as a ratio of a user's current movement distance to a user's total movement distance.

As previously described, in each expert image frame of the expert video, an expert ratio is matched in a way that corresponds to a user ratio. Accordingly, the user movement analysis part 360 calculates a user ratio for a user's current movement distance received through the communication part 340 in real time. In addition, the image controller 350 matches an expert image frame having an expert ratio matching the user ratio calculated in real time by the user movement analysis part 360 to the user's current movement distance and displays the result on the image display part 330, thereby synchronizing the expert exercise image with the user exercise image.

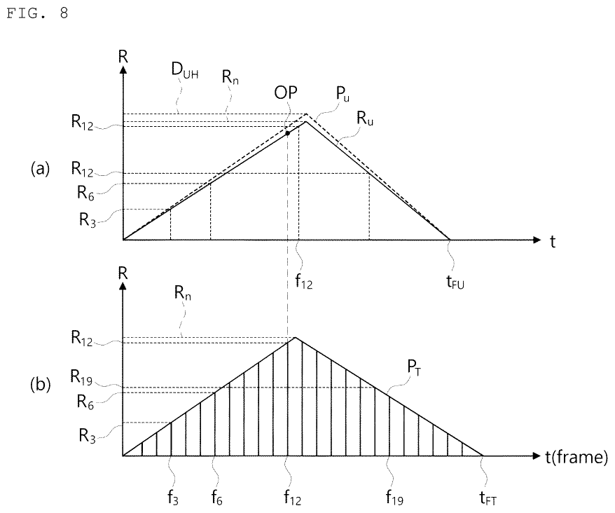

As described above, by using the expert movement ratio and the user movement ratio, as shown in FIG. 8A, the effect of synchronizing the required time t_{FT} of one exercise cycle of the expert shown in FIG. 8B with the required time t_{FU} of one exercise cycle of the user may be obtained.

Furthermore, by using the ratios, the expert's total movement distance 2D_{TH} may also be synchronized with the user's total movement distance 2D_{TH}, but synchronization is possible based on 50% of the movement distance, rather than matching the playback time.

Thus, the effect of converting the graph of the expert's movement distance over time shown in FIG. 8A into the graph (R_{U}) based on the ratio is obtained. That is, the playback time may be matched with 50% of the movement distances corresponding to each other.

In FIG. 8B, expert ratios for individual expert image frames in the expert exercise video, for example, expert ratios for expert image frames f₃, f₆, f₉, and f₁₂ are R₃, R₆, R₉, and R₁₂, respectively. As shown in FIG. 8A, the time the expert image frame with the expert ratio matching the user ratio is displayed on the image display part 330 is different from the playback time of the actual expert exercise image.

For example, among the expert image frames of the expert exercise video, f₁₂ is reproduced at the OP position when the present disclosure is not applied, whereas when the present disclosure is applied, f₁₂ is reproduced at the position of f₁₂ in FIGS. 8A and 8B, so that the expert's posture and the user's posture may be synchronized on the basis of the movement distances.

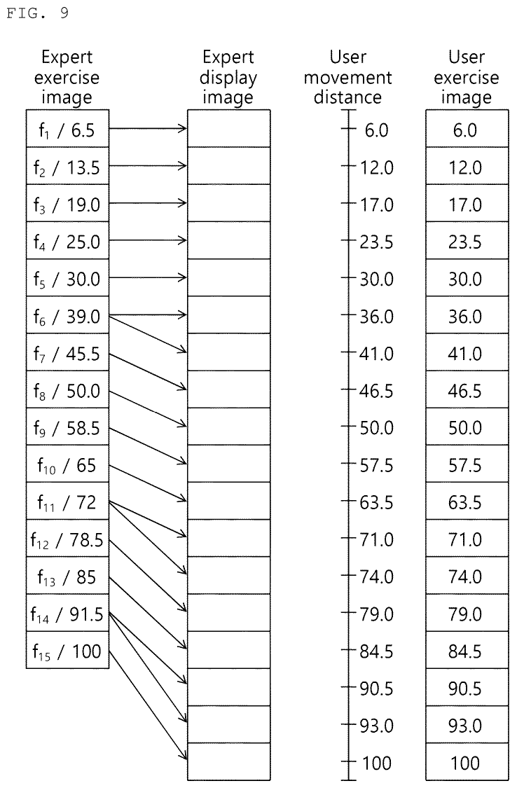

Referring to FIG. 9, a process of reproducing each expert image frame of an expert exercise video within one exercise cycle will be described.

In FIG. 9, it is exemplified that one exercise cycle in an expert exercise video is composed of 15 expert image frames, while one exercise cycle in a user exercise video is composed of 18 user image frames. That is, there is a difference between one exercise cycle of an expert and one exercise cycle of a user.

In FIG. 9, a frame order within one exercise cycle and an expert ratio are matched for each expert image frame. For example, in the case of the first frame, the expert movement distance is recorded as f1/6.5. In FIG. 9, expert display image will be synchronized as 18, the total number of frames of the user exercise image.

When a user movement distance is received in real time through the communication part 340, the user movement analysis part 360 calculates the ratio of the total movement distance of the user's one exercise cycle and the user's movement distance received in real time. For example, as shown in FIG. 9, the ratio may be calculated in real time in the order of 6.0, 12.0, etc.

At this time, the equipment controller 170 retrieves an expert ratio matching the user ratio calculated in real time by the user movement analysis part 360, for example, the expert ratio closest to the user ratio, and causes an expert image frame of the corresponding expert ratio to be displayed as an expert display image.

As an example, when the user ratio is 36.6, the expert image frame f₆ having the closest expert ratio of 39.6 may be displayed on the image display part 330. Likewise, for a user ratio of 41.0 calculated after the ratio of 36.6, the expert image frame f₆ having an expert ratio of 39.6, which is the closest, may be displayed on the image display part 330.

Through this process, it is possible to synchronize expert exercise image by matching the time required for one exercise cycle to the user exercise image in such a way that some of the expert image frames constituting the expert exercise image are displayed redundantly, or the display is omitted.

In addition, in the case of a user ratio of 50.0, which corresponds to the ratio of the movement distance, the user is in a completely standing posture during squatting, for example. At this time, since an expert image frame having a ratio of 50.0 is displayed among the expert exercise images, the expert is also in a standing posture, so that the postures during the exercise process may also be synchronized.

Meanwhile, as shown in FIG. 7, the user movement analysis part 360 according to the embodiment of the present disclosure may extract an inflection point (IP) of the user movement direction from the user movement distance. The user movement analysis part 360 may also extract one exercise cycle of the user based on the inflection point (IP) extracted from the user movement distance.

As shown in FIG. 7, a first inflection point where the movement direction changes in one direction and a second inflection point where the movement direction changes in the opposite direction to the first inflection point may be alternately extracted due to the nature of the weight training. The user movement analysis part 360 extracts an interval between a pair of adjacent first inflection points as one exercise cycle of the user.

In addition, the user movement analysis part 360 may calculate a user ratio in the current exercise cycle on the basis of the total movement distance calculated in the previous exercise cycle.

In the embodiment of the present disclosure, the user movement analysis part 360 calculates the total movement distance within the user's one exercise cycle by summing the movement distance from one first inflection point to an adjacent second inflection point, and the movement distance from that second inflection point to another adjacent first inflection point, which determine the corresponding exercise cycle.

For example, the user movement analysis part 360 calculates the deviation between the previous position of the first inflection point and the current position of the first inflection point and the deviation between the previous position of the second inflection point and the current position of the second inflection point for every exercise cycle, and then reflects the deviations in the total movement distance, thereby updating the total movement distance.

Meanwhile, exercise guide voices may be stored in the data storage part 310 of the image processing device 30 according to the embodiment of the present disclosure. At this time, an exercise guide voice may be matched with a corresponding image frame to correspond to an expert movement distance.

That is, in the embodiment of the present disclosure, the exercise guide voices are not recorded together with the expert exercise image, but the expert exercise image and exercise guide voices are stored independently, and the position of an expert image frame where a certain exercise guide voice is output is matched with an expert movement distance and stored.

In addition, when an expert exercise image is displayed on the image display part 330, that is, as described above, when the corresponding expert image frame with a ratio matching the user ratio is displayed, the image controller 350 may output the corresponding exercise guide voice in synchronization upon the display of the image frame matched with the exercise guide voice on the image display part 330.

For example, assuming that an exercise guide voice such as “Be careful not to let your knees go forward of your toes” should be output before the user fully bends his/her knees during squatting, among the expert image frames of the expert exercise video, the exercise guide voice is matched and stored with an expert image frame corresponding to the motion just before the knees are completely bent.

In this case, as previously described, in the process of synchronizing the corresponding expert image frame with the user exercise image and then reproducing, the image controller 350 outputs a matched exercise guide voice when the corresponding expert image frame is displayed on the image display part 330, so that an appropriate exercise guide voice, that is, “Be careful not to let your knees go forward of your toes” may be provided to the user right before the user fully bends his/her knees.

The image processing device 30 according to the embodiment of the present disclosure may include a sound output part 380. In the process of reproducing the expert exercise image, the image controller 350 may output the corresponding exercise guide voice together with the image through the sound output part 380 when there is a exercise guide voice matched to the corresponding expert image frame.

According to the above configuration, even if the expert exercise image is synchronized with the user exercise image because the exercise speed or movement distance is different between the expert and the user, it is possible to accurately output the voice in the required exercise posture without deformation of the exercise guide voice.

Meanwhile, as previously mentioned, in the weight training equipment 10 according to the embodiment of the present disclosure, an exercise load is provided to the pair of cable wires 120 independently. In addition, in the weight training equipment 10 according to the embodiment of the present disclosure, a user may individually perform left arm exercise and right arm exercise.

Correspondingly, the expert exercise image may include a left arm exercise image and a right arm exercise image for the same exercise type.

In addition, the image controller 350 may display either the left arm exercise image or the right arm exercise image on the image display part 330 on the basis of pulled-out cable wire information received from the weight training equipment 10 through the communication part 340.

For example, when the user performs an arm exercise using either side of the cable wire 120 using a D-grip or the like, the equipment controller 170 of the weight training equipment 10 may generate information on whether the corresponding cable wire 120 is the left or right cable wire 120 as the pulled-out cable wire information and transmit the information to the image processing device 30.

In addition, on the basis of the pulled-out cable wire information received from the weight training equipment 10 through the communication part 340, the image controller 350 may output one of the left arm exercise image and the right arm exercise image corresponding to the pulled-out cable wire information through the image display part 330.

Through this, in weight training using the left or right arm, the left and right images are reversed and displayed, so that a problem that causes confusion when the user compares exercise postures may be solved.

Although the embodiments of the present disclosure have been described with reference to the accompanying drawings, the present disclosure is not limited to the above embodiments and may be manufactured in a variety of different forms. Those skilled in the art to which the present disclosure pertains will understand that the present disclosure may be embodied in other specific forms without changing the technical spirit or essential features of the present disclosure. Therefore, it should be understood that the embodiments described above are illustrative in all respects and not limiting.

### DESCRIPTION OF THE REFERENCE NUMERALS IN THE DRAWINGS

10: weight training equipment 110: main body
120: cable wire 130: handle bar
140: equipment communication part 150: load supply module
151: first drive motor 152: second drive motor
160: position detection part 161: first position detection part
162: second position detection part 170: equipment controller
30: image processing device 310: data storage part
320: camera module 330: image display part
340: communication part 350: image controller
360: user movement analysis part 370: input part
380: sound output part

### INDUSTRIAL APPLICABILITY

The image processing device according to the embodiment of the present disclosure may be applied to a technical field of weight training equipment.

1. An image processing device interworking with weight training equipment, the image processing device comprising:
a data storage to store at least an expert exercise image;
a camera module configured to capture at least one user exercise image while on the weight training equipment;
an image display configured to display the expert exercise image and the user exercise image;
a communication device configured to receive a user movement distance based on a user's exercise from the weight training equipment; and
a controller configured to control an expert image frame to be displayed on the image display from among a plurality of expert image frames constituting the expert exercise image based on the user movement distance received through the communication device, such that an expert movement distance in the expert exercise image matches a user movement distance in the user exercise image.

2. The image processing device of claim 1, wherein the controller matches the expert exercise image and the user exercise image in units of one exercise cycle and displays the matched image on the image display.

3. The image processing device of claim 2, wherein each of the expert image frames constituting the expert exercise image includes an expert movement distance of a corresponding one of the expert image frames based on one exercise cycle of an expert.

4. The image processing device of claim 3, wherein the expert movement distance includes an expert ratio defined as a ratio of the expert movement distance of the corresponding one of the expert image frames to a total movement distance based on one exercise cycle of the expert.

5. The image processing device of claim 4, further comprising:
a user movement analyzer configured to extract one exercise cycle of the user based on the user movement distance, and
wherein the controller matches the expert exercise image and the user exercise image in units of one exercise cycle based on the one exercise cycle of the user and the one exercise cycle of the expert.

6. The image processing device of claim 5, wherein the user movement analyzer determines a total movement distance within one exercise cycle of the user, and determines a user ratio defined as a ratio of a current movement distance of the user to the total movement distance of the user, and
wherein the controller matches an expert image frame having an expert ratio matching the user ratio to the current movement distance of the user and displays the matched image on the image display.

7. The image processing device of claim 6, wherein the user movement analyzer determines a total movement distance within one exercise cycle of the user for each exercise cycle, and determines a user ratio in a current exercise cycle based on a total movement distance determined in a previous exercise cycle.

8. The image processing device of claim 6, wherein the user movement analyzer extracts an inflection point in a movement direction of the user from the user movement distance, and extracts one exercise cycle of the user based on the inflection point.

9. The image processing device of claim 8, wherein the inflection point is formed alternately with a first inflection point in which the movement direction changes in one direction and a second inflection point in which the movement direction changes in an opposite direction to the first inflection point, and
wherein the user movement analyzer extracts an interval between a pair of adjacent first inflection points as one exercise cycle of the user.

10. The image processing device of claim 9, wherein the user movement analyzer determines a total movement distance within one exercise cycle of the user by summing a movement distance from one of the pair of first inflection points to a second inflection point, and a movement distance from the second inflection point to a remaining one of the pair of first inflection points, which determine the one exercise cycle.

11. The image processing device of claim 3, wherein the data storage stores an exercise guide voice matched to a corresponding image frame to correspond to the expert movement distance, and
wherein the controller synchronizes and outputs the exercise guide voice when displaying the expert exercise image on the image display, and outputs the voice in synchronization with a display of the matched image frame on the image display.

12. The image processing device of claim 1, wherein the weight training equipment provides an exercise load based on a pair of wires, and
wherein the user movement distance is based on a pulled-out position of each of the pair of wires.

13. The image processing device of claim 2, wherein the expert exercise image includes a left arm exercise image and a right arm exercise image, and
wherein the controller displays one of the left arm exercise image and the right arm exercise image on the image display based on information of the pulled-out wire received from the weight training equipment.

14. An image processing device comprising:
a data storage to store at least a first exercise image;
a camera module configured to capture at least one user exercise image;
an image display configured to display the first exercise image and the user exercise image; and
a controller configured to control an image frame to be displayed on the image display from among a plurality of image frames constituting the first exercise image based on user movement information, such that a movement in the first exercise image corresponds to a user movement in the user exercise image.

15. The image processing device of claim 14, wherein the controller matches the first exercise image and the user exercise image in units of one exercise cycle and displays the matched image on the image display.

16. The image processing device of claim 15, wherein each of the image frames constituting the first exercise image includes movement information of a corresponding one of the image frames based on one exercise cycle.

17. The image processing device of claim 16, wherein the movement information includes first ratio defined as a ratio of the movement information of the corresponding one of the image frames to a total movement information based on one exercise cycle.

18. The image processing device of claim 14, wherein the controller is configured to extract one exercise cycle of the user based on the user movement information, and
wherein the controller matches the first exercise image and the user exercise image in units of one exercise cycle based on the one exercise cycle of the user and the one exercise cycle of the stored exercise image.

19. The image processing device of claim 18, wherein the controller determines a total movement within one exercise cycle of the user, and determines a user ratio defined as a ratio of a current movement of the user to the total movement of the user, and
wherein the controller matches an image frame having a ratio matching the user ratio to the current movement of the user and displays the matched image on the image display.

20. The image processing device of claim 19, wherein the controller determines a total movement within one exercise cycle of the user for each exercise cycle, and determines a user ratio in a current exercise cycle based on a total movement determined in a previous exercise cycle.
